(19) United States
(12) Patent Application Publication
(10) Pub. No.: US 2025/0210157 A1
(43) Pub. Date: Jun. 26, 2025

(54) SYSTEMS AND METHODS FOR ANALYZING STORED EVENTS TO DERIVE DIGITAL RESOURCE OBJECTS

(71) Applicant: Optum, Inc., Minnetonka, MN (US)

(72) Inventors: Adam RUSSELL, Arlington, MA (US); Gregory J. BOSS, Saginaw, MI (US); Ramprasad Anandam GADDAM, Gurgaon (IN); Robert Kahn ROSSMILLER, Brookfield, WI (US)

(21) Appl. No.: 18/392,353

(22) Filed: Dec. 21, 2023

(51) Int. Cl. G16H 10/60; G06Q 40/04

(52) U.S. Cl. CPC G16H 10/60; G06Q 40/04; G06Q 2220/00

(57) ABSTRACT

A computer-implemented method includes detecting, by one or more processors, a presence of an event written to a blockchain as one or more data objects, the event being associated with an entity; determining, by the one or more processors, one or more digital resource object categories to be mapped to the event based on one or more factors; determining, by the one or more processors, at least one magnitude value associated with each of the one or more digital resource object categories based on an evaluation of the event, the evaluation including an analysis of one or more parameters associated with the event; and generating, by the one or more processors, an aggregate digital resource object based on each determined magnitude value.

### TECHNICAL FIELD

The present disclosure relates generally to the field of data analytics, storage, and cryptography. In particular, the present disclosure relates to analyzing data objects securely stored in distributed ledgers and generating digital resource objects based on the analysis.

### BACKGROUND

Distributed ledger technology (e.g., blockchain) may enable the functionality of digital currencies by serving as a decentralized and secure system for recording and verifying transactions. In various industries, distributed ledger technology has been used to record various types of events and transactions. However, without adequate incentives for industry systems and entities to store such data in these data stores, the amount of data that is provided can be limited. Additionally, useful application of the technology surrounding digital currencies is currently limited in various industry sectors, although implementation of such technology can lead to numerous benefits and improvements.

The present disclosure is directed to overcoming one or more of the above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

In some aspects, the techniques described herein relate to a computer-implemented method, including: detecting, by one or more processors, a presence of an event written to a blockchain as one or more data objects, the event being associated with an entity; determining, by the one or more processors, one or more digital resource object categories to be mapped to the event based on one or more factors; determining, by the one or more processors, at least one magnitude value associated with each of the one or more digital resource object categories based on an evaluation of the event, the evaluation including an analysis of one or more parameters associated with the event; and generating, by the one or more processors, an aggregate digital resource object based on each determined magnitude value.

In some aspects, the techniques described herein relate to a computer-implemented method, wherein the event is written to the blockchain based on a consensus mechanism.

In some aspects, the techniques described herein relate to a computer-implemented method, wherein the one or more digital resource object categories include one or more health-related categories.

In some aspects, the techniques described herein relate to a computer-implemented method, wherein the one or more factors include: a predefined mapping of known events to the one or more digital resource object categories; and/or an existing association of the event to the one or more digital resource object categories based on information available in a public database.

In some aspects, the techniques described herein relate to a computer-implemented method, wherein the one or more parameters include at least one of an extent or a duration associated with the event.

In some aspects, the techniques described herein relate to a computer-implemented method, wherein determining the at least one magnitude value further includes: for a respective digital resource object category mapped to the event, determining, by the one or more processors, one or more digital resource object sub-categories mapped to the respective digital resource object category; determining, by the one or more processors, at least one parameter value associated with each of the one or more digital resource object sub-categories based on an evaluation of a data association between the respective digital resource object category and each of the one or more digital resource object sub-categories; and aggregating, by the one or more processors, each determined parameter value.

In some aspects, the techniques described herein relate to a computer-implemented method, wherein the one or more digital resource object sub-categories are associated with a mortality projection model or a survival analysis model.

In some aspects, the techniques described herein relate to a computer-implemented method, wherein the one or more digital resource object categories are associated with a health determinant model.

In some aspects, the techniques described herein relate to a computer-implemented method, wherein determining the at least one magnitude value further includes: receiving, by the one or more processors, one or more attribute data objects associated with the entity; determining, by the one or more processors, whether each of the one or more attribute data objects is a positive or a negative modifier of the evaluation of the event; and modifying, by the one or more processors, the at least one magnitude value based on the determination.

In some aspects, the techniques described herein relate to a computer-implemented method, wherein the event includes a plurality of sub-events detected over a period of time, and wherein determining the at least one magnitude value further includes: for a respective digital resource object category mapped to the event, determining, by the one or more processors, a data completeness score associated with the event, the data completeness score being based on a data completeness rating of the plurality of sub-events detected over the period of time; and modifying, by the one or more processors, the at least one magnitude value based on the data completeness score.

In some aspects, the techniques described herein relate to a computer-implemented method, wherein a value of the aggregate digital resource object is modified by a fluctuating weight, the fluctuating weight being determined based on a quantity of similarly classified data objects detected in the blockchain as compared to the one or more data objects.

In some aspects, the techniques described herein relate to a computer-implemented method, further including mapping, by the one or more processors, the entity to a group entity based on: a requirement to include the group entity in the blockchain with the event; or a search of a public or private repository that includes information related to the group entity.

In some aspects, the techniques described herein relate to a computer-implemented method, further including issuing, by the one or more processors, the aggregate digital resource object to the group entity or to the entity.

In some aspects, the techniques described herein relate to a computer-implemented method, wherein: the aggregate digital resource object is used to reduce a plan premium; or the aggregate digital resource object is exchangeable on an exchange platform for one or more other digital resource objects.

In some aspects, the techniques described herein relate to a computer-implemented method, further including: generating, by the one or more processors, a digital resource object for each digital resource object category mapped to the event.

In some aspects, the techniques described herein relate to a system, including: at least one memory having processor-readable instructions stored therein; and at least one processor configured to access the at least one memory and execute the processor-readable instructions to perform operations, the operations including: detecting a presence of an event written to a blockchain as one or more data objects, the event being associated with an entity; determining one or more digital resource object categories to be mapped to the event based on one or more factors; determining at least one magnitude value associated with each of the one or more digital resource object categories based on an evaluation of the event, the evaluation including an analysis of one or more parameters associated with the event; and generating an aggregate digital resource object based on each determined magnitude value.

In some aspects, the techniques described herein relate to a system, wherein the event is written to the data store based on a consensus mechanism.

In some aspects, the techniques described herein relate to a system, wherein determining the at least one magnitude value further includes: for a respective digital resource object category mapped to the event, determining one or more digital resource object sub-categories mapped to the respective digital resource object category; determining at least one parameter value associated with each of the one or more digital resource object sub-categories based on an evaluation of a data association between the respective digital resource object category and each of the one or more digital resource object sub-categories; and aggregating each determined parameter value.

In some aspects, the techniques described herein relate to a system, wherein the one or more digital resource object categories are associated with a health determinant model, and wherein the one or more digital resource object sub-categories are associated with a mortality projection model or a survival analysis model.

In some aspects, the techniques described herein relate to a non-transitory computer-readable medium storing a set of instructions that, when executed by at least one processor, cause the at least one processor to perform operations including: detecting a presence of an event written to a blockchain as one or more data objects, the event being associated with an entity; determining one or more digital resource object categories to be mapped to the event based on one or more factors; determining at least one magnitude value associated with each of the one or more digital resource object categories based on an evaluation of the event, the evaluation including an analysis of one or more parameters associated with the event; and generating an aggregate digital resource object based on each determined magnitude value.

It is to be understood that both the foregoing general description and the following detailed description contain examples and are explanatory only and are not restrictive of the disclosed embodiments, as claimed.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 depicts an example system infrastructure for deriving digital resource objects, according to one or more embodiments.

FIG. 2 depicts an example method of a flowchart for generating digital resource objects based on event data, according to one or more embodiments.

FIG. 3 depicts another example method of a flowchart for generating digital resource objects based on event data, according to one or more embodiments.

FIG. 4 depicts an example implementation of a computer system that executes techniques presented herein, according to one or more embodiments.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description contain examples and are explanatory only and are not restrictive of the features, as claimed.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as, “substantially” and “generally,” are used to indicate a possible variation of ±10% of a stated or understood value.

Various embodiments of the present disclosure relate generally to systems and methods for analyzing stored events to derive digital resource objects, and more particularly, to systems and methods for analyzing data objects securely stored in distributed ledgers and generating digital resource objects based on the analysis.

As discussed previously, distributed ledger technology, such as blockchain, may enable the functionality of digital currencies by serving as a decentralized and secure system for recording and verifying transactions. In various industries, distributed ledger technology has been used to record various types of events and transactions. However, there is a lack of adequate incentives for industry systems and entities to store such data in distributed ledgers such as a blockchain, which can lead to the amount of data that is provided being limited. Additionally, useful application of the technology surrounding digital currencies is currently limited in various industry sectors, although implementation of such technology can lead to numerous benefits and improvements

As discussed herein, digital resource objects may include digital coins, digital tokens, digital currencies, and the like, which may derive value from receipt of data from various entities (e.g., associations, groups, organizations, or companies subscribing to a health insurance providers) and/or their members. In one or more embodiments, digital resource objects may be exchangeable for other digital resource objects or hard currencies. Before a detailed description of the various incentive systems for deriving digital resource objects are described according to one or more embodiments, a general summary of how data (e.g., health data) provided by entities may be written to a blockchain is provided below.

Trustfully recording user data of entities, such as data including health events of individuals or groups, in distributed ledgers are not well known in industry today. For example, there is a lack of an adequate consensus mechanism to trustfully record user data of individuals or groups. Known consensus mechanisms such as Proof of Work, Proof of Stake, Delegated Proof of Stake, Proof of Capacity, Proof of Coverage, Proof of Elapsed Time, Proof of Authority, Proof of Activity, among others, do not currently provide a way to verify health events.

According to one or more embodiments, a Proof of Health (PoH) or a Proof of Event (PoE) consensus mechanism enables user data to be written to a blockchain in a secure and trustable manner based on occurrences of certain events (e.g., health-related or other types of events) that have been verified by one or more records. For example, a health event that has occurred for an entity, such as events related to exercise, diet, diagnosis history, etc., is verified or validated based on a system of miners and validators that may verify that an occurred event is recorded in a corresponding system (e.g., health records, medical records, etc.). Additionally, the verification includes performing a check on whether the attributes of an occurred event match the attributes of the event recorded in the corresponding system. If an occurred event is recorded and the attributes match, the occurred event passes a validation/verification check.

As a subsequent step, the PoH and/or PoE consensus mechanism employs a consensus threshold check, where it is determined whether an occurred event has met a threshold number of validations. The threshold number is predetermined and may vary depending on characteristics of an occurred event. If an occurred event is determined to have met a threshold number of validations or determined to have surpassed a number of validations or a consensus threshold, the occurred event is written to a blockchain or other forms of distributed ledgers such as Hashgraph and Directed Acyclic Graphs (DAGs), among others. If an occurred event is determined to not have met or surpassed a threshold number of validations, the occurred event may be added to a secondary validation queue for further verification. The PoH and/or PoE consensus mechanism enables all participants in the described system to be miners and/or validators. Some validators may contribute more to reaching a validation threshold more than others.

However, without an incentive system or a reward mechanism, entities may not contribute data consistently. Thus, one or more embodiments of the present disclosure relate to various methods and systems for deriving digital resource objects based on an incentive system that provides rewards to entities or their members for contributed data. Although embodiments of the present disclosure are described in relation to health-related events (“health events”) and health-related data (“health data or event data”), it should be noted that the present disclosure is not limited to such data. For example, the embodiments of the present disclosure is applicable to other types of events in other types of industries where contributed data is valuable. Additionally, although embodiments of the present disclosure is described with respect to blockchain, the described embodiments is compatible with other forms of distributed ledger technologies, such as Hashgraph and DAGs, among others.

According to one or more embodiments, digital tokens are generated based on event data submitted by entities or their members that correspond to certain health events. The digital tokens include various types of digital tokens that represent various health determinants such as fitness, diet choices, subjective sense of health and well-being, diagnosis history, prescription (Rx) adherence, among others. Each of these health determinants are mapped to a digital token category, and a digital token is generated for each digital token category. For example, based on characteristics of a health event that is written to a distributed ledger using a consensus mechanism (e.g., PoH and/or PoE consensus mechanism), one or more digital token categories are mapped to the health event. Each digital token may be represented by or mapped (e.g., coded) to different colors such as purple, red, blue, etc. For example, a red coin may represent fitness habits, a purple coin may represent dietary habits, and so forth.

Each digital token mapped to a digital token category derives a corresponding value or magnitude based on an evaluation or analysis of an event. For example, the event may correspond to a fitness event, a dietary event, a diagnosis event, and so forth, and an extent or duration of the event may be analyzed to determine an impact to the entity's mortality projection or life expectancy model. Based on a magnitude of an event's impact to the entity's mortality projection or life expectancy model, a value is generated for each digital token mapped to a digital token category. The mechanisms on how a value of a digital token is generated is discussed further in detail with respect to the discussion of the figures below.

As one skilled in the art will appreciate in light of this disclosure, the embodiments disclosed herein achieve certain technical advantages and improvements, including but not limited to the following: (1) techniques described in the present disclosure improve the field of computing, especially in the field of computing as applied to distributed ledger technology, by analyzing data objects securely stored in a distributed ledger to generate digital tokens and assign values to digital tokens based on analysis of certain events; (2) additionally, the techniques described in the present disclosure improve the applicability, efficiency, functioning, and security of computing systems by using and improving upon distributed ledger technology, particularly in the context of the healthcare industry where previous applications of distributed ledger technology were limited, by providing a consensus mechanism to trustfully and securely store event data, and by providing incentive and reward mechanisms for entities to continue providing event data to a computing system in exchange for digital tokens (e.g., the reward), where the value of digital tokens may be computed based on an analysis of the submitted event data, the value of digital token indicative of certain attributes or characteristics of an entity associated with the digital token; and (3) techniques described in the present disclosure are inherently rooted in computer technology in order to overcome limited applications of distributed ledger technology as applied to the healthcare industry, by providing efficient ways to analyze data objects securely stored in a distributed ledger and generate one or more digital tokens based on the analysis, and providing unconventional ways to use the digital tokens for entities or members who provide the data, thereby providing new and useful ways of using distributed ledger technology and also improving upon computer resource utilization (e.g., memory consumption, processor utilization, network transfer, etc.) tied to the use of distributed ledger technology for performing the various operations and functions described herein.

The disclosed embodiments offer technical advantages over existing consensus mechanisms in the field of distributed ledger technology such as reduced computing power and reduced computing time. Additionally, the disclosed embodiments offer reduced green gas emission and enable democratic participation for participants to receive rewards without placing harsh financial burdens on each participant. The technical advantages and improvements discussed above are not the sole advantages. Other technical advantages and improvements may be apparent to one of ordinary skill in the art.

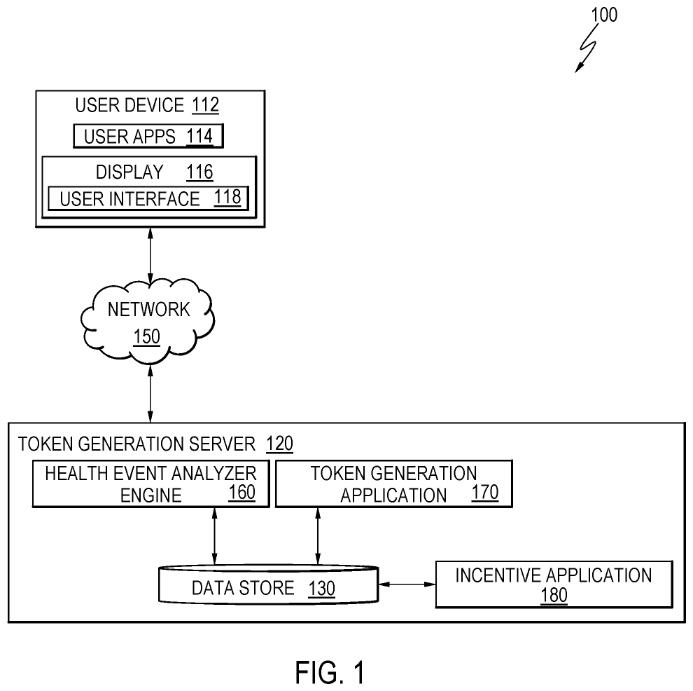

Referring now to the appended drawings, FIG. 1 depicts an example system infrastructure for deriving digital resource objects, according to one or more embodiments. The system 100 may include a user device 112 and a token generation server 120, which are in data communication with each other via a network 150. The network 150 includes, for example, the Internet, intranets, extranets, wide area networks (WANs), local area networks (LANs), wired networks, wireless networks, cable networks, satellite networks, or other suitable networks, etc., or any combination of two or more such networks.

The token generation server 120 includes, for example, a server computer or any other system providing computing capability for the generation of digital coins, digital tokens, digital currencies, among others. Alternatively, the token generation server 120 employs a plurality of computing devices that may be arranged, for example, in one or more server banks or computer banks or other arrangements. Such computing devices may be located in a single installation or may be distributed among many different geographical locations. For example, the token generation server 120 includes a plurality of computing devices that together may comprise a hosted computing resource, a grid computing resource, and/or any other distributed computing arrangement. These plurality of computing devices are often referred to as nodes, which is used to host one or more distributed ledgers as discussed in the present disclosure. In some cases, the token generation server 120 corresponds to an elastic computing resource where the allotted capacity of processing, network, storage, or other computing-related resources may vary over time. The token generation server 120 may further include a scalable cloud computing environment or platform with scalable resources for computations and/or data storage.

Various applications and/or other functionality are executed in the token generation server 120 according to one or more embodiments. Also, various data is stored in data store 130 that is accessible to the token generation server 120. The data store 130 may be representative of a plurality of data stores as can be appreciated. The data store 130 includes one or more distributed ledgers (e.g., blockchain, Hashgraph, DAGs, etc.), which are hosted across the nodes of the token generation server 120, as described above. The data stored in the data store 130, for example, is associated with the operation of the various applications and/or functional entities described below.

The components executed in the token generation server 120, for example, include a health event analyzer engine 160, a token generation application 170, an incentive application 180, and other applications, services, processes, systems, engines, or functionality not discussed in detail herein. The health event analyzer engine 160 is executed for detection of event data (e.g., health events) written to the data store 130 and analysis of the event data to derive one or more digital tokens for a member of an associated entity who is providing the data.

The token generation application 170 is executed for setting or modifying the parameters for the generation of digital tokens for a member based on the analysis performed by the health event analyzer engine 160. The token generation application 170 may further be responsible for issuance of the one or more generated digital tokens to a member. As discussed above, data is written to the data store 130 based on provided data from the associated entity via a consensus mechanism such as the PoH and/or PoE consensus mechanism. The data provided to the data store 130 corresponds to health events experienced by an associated entity or a member who may be a participant of the incentive system discussed herein.

The incentive application 180 is be executed for providing an electronic network platform to an associated entity or a member of an associated entity for using or exchanging the generated digital tokens. For example, an associated entity or a member may exchange the generated digital tokens for other types of digital tokens, publicly trade or internally trade the generated digital tokens for other digital currencies, use the generated digital tokens to lower health premium, determine optimal health plans, and/or offset premium payments via the incentive application 180.

The user device 112 is representative of a plurality of user devices that is coupled to the network 150. The user device 112 includes, for example, a processor-based system such as a computer system. Such a computer system may be embodied in the form of a desktop computer, a laptop computer, personal digital assistants, cellular telephones, smartphones, tablet computer systems, or other devices. The user device 112 includes a display 116. The display 116 may include, for example, one or more devices such as liquid crystal display (LCD) displays, gas plasma-based flat panel displays, organic light emitting diode (OLED) displays, electrophoretic ink (E ink) displays, or other types of display devices, etc.

The user device 112 is configured to execute various applications such as a user application 114 and/or other applications. The user application 114 may be executed in the user device 112, for example, to access network content provided by the token generation server 120 and/or other servers, thereby rendering a user interface 118 on the display 116 of the user device 112. To this end, the user application 114 includes, for example, a browser, a dedicated application, etc., and the user interface 118 may include a network page, an application screen, etc.

An associated entity or a member who is a participant of the disclosed incentive system may use the user device 112 to provide health event data (“event data”) to the data store 130 via the user application 114. The associated entity includes, for example, associations, groups, organizations, or companies that are affiliated with a health insurance provider (e.g., subscribed to health insurance from the health insurance provider). In some embodiments, the associated entity or its members continuously provide event data to the data store 130, provide event data upon occurrences of certain health events, or provide event data at scheduled times that are predetermined. The user device 112 is configured to access other devices, server, or data stores connected via the network 150, in order to access, retrieve, and provide event data to the token generation server 120 or the data store 130 thereof. In some embodiments, the associated entity or a member further executes the user application 114 to view a progress, status, or quantity of digital tokens generated for the associated entity. Additionally, in some embodiments, the associated entity or member executes the user application 114 to use or trade generated digital tokens for various incentives, as hosted by the incentive application 180.

Although some of the operations and functionality of each of the health event analyzer engine 160, the token generation application 170, and the incentive application 180 are described above, it should be noted that one or more of the health event analyzer engine 160, the token generation application 170, or the incentive application 180 may provide the operation and functional capabilities of any individual application or engine of the token generation server 120. That is, in some embodiments, the health event analyzer engine 160 is configured to provide the operations and functionality of the token generation application 170 and/or the incentive application 180, the token generation application 170 is configured to provide the operations and functionality of the health event analyzer engine 160 and/or the incentive application 180, and the incentive application 180 is configured to provide the operations and functionality of the health event analyzer engine 160 and/or the token generation application 170. The above components of the system 100 are implemented in hardware, firmware, software, or a combination thereof.

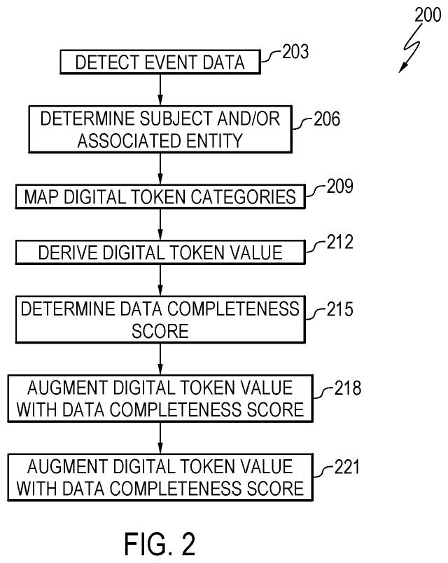

FIG. 2 depicts an example method 200 for generating digital resource objects based on event data, according to one or more embodiments. The method 200 is an example method for analyzing event data (e.g., health events) written to the data store 130 and generating one or more digital tokens for issuance to an associated entity or its members. The token generation server 120 is configured to implement the method 200 according to various embodiments.

At step 203, the health event analyzer engine 160 is configured to detect event data written to the data store 130. As discussed above, event data corresponds to health events associated with an entity or a member who is a participant of the disclosed incentive system. An associated entity or a member who is a participant of the disclosed incentive system includes organizations, companies, associations, groups, or any combinations thereof, who are affiliated with a health insurance provider (e.g., subscribed to a health insurance from the health insurance provider). For example, members of an associated entity may be an employee of a company or a member of a group that is subscribed to a health insurance from a health insurance provider.

Event data includes data corresponding to a workout event, a dietary event, a diagnosis event, a medication prescription event, and other health events for a member of an associated entity. For example, in various embodiments, a member provides, via the user device 112, a particular exercise event or a fitness event such as how many steps he or she walked for the day. The member may provide such data directly via the user device 112, or an associated entity affiliated with the member may provide that data via the user device 112. Other examples of event data includes events such as meals and specific meal items consumed, specific prescription medications prescribed, and other health events that may be quantified in some way. In some embodiments, associated entities or its members provide event data to the data store 130 at specific intervals or upon occurrences of certain events. As will be discussed, in some embodiments, compliance in adequately submitting event data is factored in to how many digital tokens an associated entity or its members receive. Event data that is submitted is written to the data store 130 based on one or more consensus mechanisms, such as the PoE, PoH, or other consensus mechanisms described above.

At step 206, the health event analyzer engine 160 is configured to determine a subject and/or an associated entity that submitted event data corresponding to a particular event. For a particular event that was written to the data store 130, the health event analyzer engine 160 determines who is the subject (e.g., member, employee, etc.) of the particular event and what associated entity he or she is associated with. The health event analyzer engine 160 is configured to assign to the particular event identification parameters that identify who the subject is and the determined associated entity affiliated with the subject. The health event analyzer engine 160 is configured to search a public or private repository to identify the subject and the associated entity. In some embodiments, the health event analyzer engine 160 requires that identifying parameters of the associated entity be entered by a subject member when submitting event data via a smart contract. In some embodiments, smart contract functionality is implemented for a distributed ledger corresponding to the data store 130, and parameters of the smart contract functionality is set by the health event analyzer engine 160.

At step 209, the health event analyzer engine 160 determines one or more digital token categories to be mapped to a particular event corresponding to submitted event data. Digital token categories include one or more of health-related categories such as adherence, fitness, diet choices, subjective sense of health and well-being (SSH&WB), diagnosis history, and the like. Digital token categories are mapped to a particular event by predefining known health events with certain categories. For example, activities like walking, playing sports, running, or weight lifting are mapped to the digital token category “fitness.” In another example, meal events that are included in submitted event data may contain data relating to particular food items consumed, quantities of certain items consumed, food types consumed, etc. Meal events are mapped to the digital token category “diet choices.” It should be noted that particular events may be mapped to greater than one digital token category depending on the nature and characteristics of the event. For example, fitness activities and meal events may also both be additionally mapped to the SSH&WB digital token category. For each digital token category mapped to a particular event, an individual digital token is generated. In the example above for a particular meal event, the health event analyzer engine 160 is configured to generate a diet choices digital token and a SSH&WB digital token. Each generated digital token may be coded with different identification parameters within the token generation server 120. In some embodiments, a generated digital token is represented by colors (e.g., purple, red, blue, etc.).

In some embodiments, the health event analyzer engine 160 is configured to search for associations between certain categories and particular events from public sources such as the National Institutes of Health (NIH), the World Health Organization (WHO), or similar sources. In other embodiments, associated entities define which digital token categories are mapped to certain events, or define new digital token categories that are applicable to their members.

Referring now to step 212, the health event analyzer engine 160 is configured to determine a value of each digital token that is generated. After determining the number and type of concurrent digital token categories mapped to a particular event, the health event analyzer engine 160 analyzes an extent or duration of the particular event corresponding to the submitted event data. For example, a duration of a particular exercise event (e.g., 5 minutes vs. 30 minutes of exercise) could be a determining factor in the amount of digital tokens calculated.

In some embodiments, the health event analyzer engine 160 considers a personal attribute modifier in determining how much impact a particular event may have on a member. For example, a daily 30-minute walk on a treadmill may have more health benefits to someone who is morbidly obese than someone who is in excellent shape. In another example with a first member with Type 1 diabetes and a second member with Type 2 diabetes, a particular meal event that regularly reduces saturated and trans fats would be more beneficial to the member with Type 2 diabetes. A personal attribute modifier increases or decreases the effect or impact a particular event has in the number of digital tokens earned for a member, for a particular event.

An in-depth example on how the health event analyzer engine 160 determines the value of digital tokens that are generated is described below with respect to Table 1 below.

TABLE 1
Example Effects on Digital Token Categories and Sub-Categories

| Example | Summary metrics | | | |
|---|---|---|---|---|
| Entries | | Healthy years | Quality-Adjusted Life | |
| Category | Life expectancy | of life | Expectancy | . . . |
| Fitness | History of Endurance | +15 | +15 | |
| | workouts +6 | | | |
| Diet Choices | +3 | +10 | +10 | |
| Subj sense of | +3 | +4 | +56.8 (maximum) | |
| H&WB | | | | |
| Diagnosis History | Breast Cancer: −5 | High BP: −2 | Depression −12 | |
| Rx Adherence | . . . | . . . | . . . | |

In Table 1 above, the values in each cell are derived from peer-reviewed articles describing an action and its effect on one or of the summary metrics described above. Each row corresponds to a digital token category as discussed above. Each column corresponds to a digital token sub-category associated with a mortality projection model or a survival analysis model of a member. In some embodiments, a mortality projection model or a survival analysis model includes a life expectancy projection, a healthy years of life projection, a quality-adjusted life expectancy projection, and/or other types of health projections of a member associated with a particular event being analyzed by the health event analyzer engine 160. Numbers in each cell describe the effect of the row's category on a column's category and is referred to herein as the “effect value”. For example, for particular events corresponding to a history of endurance workouts for a member, such event data is estimated to have a net plus 6 years on a life expectancy projection of a member and estimated to have a net plus 15 years on healthy years of life and quality-adjusted life expectancy projection of a member. In example literature, a history of endurance workouts is described as working out for more than 30 minutes at least three times per week.

Still referring to the above example of endurance workouts, to derive the effect of one endurance workout event on a quantity of fitness digital tokens generated, the following equation is relied upon:

$$1 run ( \frac{1 week}{3 runs} ) ( \frac{1 year}{52 weeks} ( + 6 effect ) ) \\ ( \frac{1 Fitness Coins}{1 year} ) = 0.0 \; 384 Fitness coins \qquad Eq . 1$$

The above equation calculates a quantity or magnitude of fitness digital tokens generated for one 30-minute run based on the event's effect on the “life expectancy” sub-category. For the sake of this example, history is limited to one year (52 weeks), and 0.0384 fitness tokens are generated. As mentioned above, example literature describes that a history of endurance workouts has a net plus 6 years (e.g., effect value) on a person's life expectancy projection, and this value is factored in to Equation 1. The negative effects of not exercising is also be found in literature describing how a lack of exercise may affect one of the digital token sub-categories (life expectancy projection, healthy years of life projection, quality-adjusted life expectancy projection, etc.). Thus, a lack of exercise data may affect the effect values that other mapped categories, such as the SSH&WB category (since SSH&WB may be affected by fitness events), have on each mapped sub-category, for example.

In another example involving the diet choices digital token category, the equation to determine the value of the diet choices digital token may be parameterized by the balance of a member's diet. For example, literature may describe eating more legumes having a positive effect on a person's healthy years of life. Thus, a meal event with beans as the main protein could have the following effect on a value of a diet choices digital token based on the following equation:

$$1 meal ( \frac{1 w k}{18 meals} ) ( \frac{1 yr}{52 w k} ( + 10 effect ) ) \\ ( \frac{1 Diet Choice Coins}{1 yr} ) = 0.0 \; 092 Diet Choice coins \qquad Eq . 2$$

In this example involving equation 2, example literature may describe a history of eating legumes having a net plus 10 years (e.g., effect value) on a person's healthy years of life projection, and this value is factored into equation 2. Additionally, for the sake of this example, history is limited to 18 meals, which is described to include a majority of meals in the week having beans included in the meals. For this example, the health event analyzer engine 160 may generate 0.0092 diet choice tokens for one meal event with beans as the main protein.

It should be noted that equation 1 and equation 2 are only provided as examples for understanding the embodiments of the present disclosure. These examples only factor in the fitness and diet choices digital token category and its individual impact on a life expectancy projection and a healthy years of life projection, respectively. In various embodiments, a mapped digital token category may impact more than one digital token sub-category. In such cases, a value may be derived for each effect the digital token category may have on each mapped digital token sub-category, and all resulting individual effect values may be augmented for each digital token category. If there is no available data in peer reviewed articles or example literature for a specific digital token sub-category (e.g., no data available regarding meal event on a life expectancy projection), the unavailability of data would not add or detract from the value of diet choices digital tokens generated.

Additionally, the above examples of effect values are based on methods and data provided by example literature. An associated entity or a health insurance provider may customize the parameters that a particular mapped digital token category may have on a particular digital token sub-category, based on the nature and characteristics of a particular event and/or other factors.

At step 215, the health event analyzer engine 160 is configured to determine a data completeness score for each mapped digital token category. The data completeness score is based on a grading scale for timely or consistent contribution of event data. For example, Table 2 below and the Base-Grading scale describe a data completeness score for a member who contributes event data.

TABLE 2
Data Completeness Table

| | | | | Subj | |
|---|---|---|---|---|---|
| | | | Diet | sense of | Diag |
| Person A | Adherence | Fitness | Choices | H&WB | History |
| Day 1 | | No activity | Missing | | |
| Day 2 | Missing | No activity | | | |
| Day 3 | | No activity | Missing | | |
| Day 4 | | No activity | Missing | | |
| Day N | | No activity | | | |
| Completeness | B | A | D | A | A |
| Score/Coin | | | | | |
| Quality Score | | | | | |

The Base-Grading scale, for example, includes grades of: 99-95: A—(1.0, complete), 94-80: B—(0.75, mostly complete), 79-70: C—(0.5, gaps in data), 69-60: D—(0.25, missing), and <60: F—(0, incomplete). The Base-Grading scale includes augment values such as 1.0 for an A grade, a 0.75 for a B grade, a 0.5 for a C grade, and so forth. Each of these augment values is used to determine the total value of digital tokens an associated entity or a member receives for each digital token category. Additionally, the parameters of determining the data completeness score may be different for each digital token category based on the nature and/or characteristics of event data that is submitted. For example, a member's data completeness score for the fitness category may not be penalized for missing data, as there is either a fitness event written to the data store 130 or not. Meals on the other hand, are affected by their completeness score because everyone must eat. Thus, the health event analyzer engine 160 is configured to determine if a member did not report a meal (assuming a member records meals that were missed or skipped).

At step 218, the digital token values derived at step 212 are augmented with the data completeness scores determined at step 215. For example, referring to the example described with respect to equation 2, if a member recorded 80% of his or her own meals within the last year, the corresponding data completeness score for the diet choices digital token category corresponds to a B (augment value of 0.75 according to the Base-Grading scale). The augment value of 0.75 may be used to augment the digital token values derived at step 212. Thus, the member would receive 0.0092*0.75=0.0069 diet choices digital tokens in this example.

Further examples of the data completeness score and how it is used to augment the digital token values derived at step 212 are described below with respect to Table 3 and equation 3 below:

TABLE 3
Aggregate Digital Token Values based on Completeness Grade

| | Summary metrics | | | |
|---|---|---|---|---|
| 1. Person A | Life | Healthy | Completeness | Aggregate Score |
| Segment | Expectancy | Years of Life | Grade | for Segment |
| Adherence | 22 | 16 | A (1.0) | 38 Adherence coins |
| Fitness | 43 | 77 | D (0.25) | 30 Fitness coins |
| Diet Choices | 99 | 89 | C (0.50) | 95 Diet Coins |
| Subj Sense of | 34 | 90 | B (0.75) | 93 HWB Coins |
| H&WB | | | | |
| Diagnosis | 22 | 67 | A (1.0) | 89 Dx History Coins |
| History | | | | |
| Aggregate | — | — | — | 345 Health Coins |
| score | | | | (aggregate) |

$$Segment ( Coin ) Score = Sum ( rows ) \cdot completeness score \qquad Eq . 3$$

Table 3 above lists example digital token values for each digital token category and associated sub-categories. The effect that each digital token category has on each sub-category (e.g., effect values of 22 and 16 for the effect the “adherence” category has on “life expectancy projection” and “healthy years of life projection” sub-categories, respectively) is determined according to equations 1 and 2 described above.

To obtain an aggregate score for each digital token category, equation 3 is used to aggregate all individual effect values for corresponding sub-categories. Referring still to the adherence category example, the effect values of 22 and 16 are aggregated, which would result in an aggregate effect value of 38. The aggregate effect value of 38 is multiplied by the augment value of 1.0 (corresponding to Base-Grading scale grade of A), which would result in an aggregate score of 38 for the adherence category.

Describing another example from Table 3 above, the effect values of 43 and 77 for the fitness category are aggregated to obtain an aggregate effect value of 120. The aggregate effect value of 120 is multiplied by the augment value of 0.25 (corresponding to Base-Grading scale grade of D), which would result in an aggregate score of 30 for the fitness category. The aggregate scores is used to generate the correct quantities of digital tokens corresponding to each digital token category. For example, the health event analyzer 160 or the token generation application 170 is configured to generate 38 adherence digital tokens, 30 fitness digital tokens, 95 diet choices digital tokens, 93 SSH&WB digital tokens, and 89 diagnosis history digital tokens. Each of the aggregate scores may further be aggregated to generate a total aggregate value of digital tokens (e.g., health tokens or health coins). For example, the aggregate scores of 38, 30, 95, 93, and 89 are aggregated to generate 345 aggregate digital tokens for a member. In some embodiments, the health event analyzer engine 160 is configured to generate the aggregate digital tokens or individual digital tokens generated for each digital token category. In other embodiments, the token generation application 170 is configured to generate the aggregate digital tokens or individual digital tokens generated for each digital token category

At step 221, the aggregate digital tokens determined for a member in step 218 is augmented by an optional weight, described by equation 4 below:

Total(Pop Health Score)=Sum(Segment Sore)·(optional weights) Eq. 4:

The optional weights may fluctuate based on the amount of data corresponding to a particular digital token category that is stored in the data store 130. More populated data is weighted less heavily, which can correspond to the digital coin of that category having less value. Data which is scarcer in the data store 130 is weighted more heavily, mimicking supply and demand feedback.

Although the method 200 is described to be executed by the health event analyzer engine 160 and/or the token generation application 170, the embodiments of the present disclosure are not limited thereto. For example, in some embodiments the token generation application 170 is configured to execute each of the steps for method 200. In other embodiments, the incentive application 180 is configured to execute one or more of the steps of method 200 depending on one or more factors associated with a provider who may be generating digital tokens for associated entities and their members.

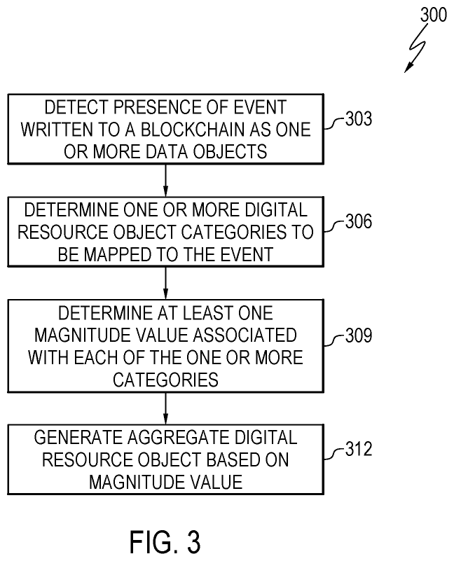

FIG. 3 depicts another example method 300 of a flowchart for generating digital resource objects based on event data, according to one or more embodiments. The method is similar to method 200, and certain steps of method 200 correspond to the steps described for method 300. However, not all corresponding steps of method 200 are described for method 300 for the sake of brevity. For example, an individual step of method 300 may encompass one or more individual steps of method 200. The method 300 describes steps for analyzing event data (e.g., health events) written to the data store 130 and generating one or more digital tokens for issuance to an associated entity or its members. The token generation server 120 is configured to implement the method 300 according to various embodiments. Beginning with step 303, the token generation server 120 is configured to detect a presence of an event written to a blockchain as one or more data objects. The event may be associated with an entity or a member of an entity. In some embodiments, the event is written to a blockchain based on a consensus mechanism.

At step 306, the token generation server 120 is configured to determine one or more digital resource object categories to be mapped to the event based on one or more factors. The one or more digital resource object categories include one or more of: adherence, fitness, diet choices, subjective sense of health and well-being, or diagnosis history, among others. In some embodiments, the one or more digital resource object categories are associated with a health determinant model. The one or more factors include detection of a predefined mapping of known events to the one or more digital resource object categories and/or detection of an existing association of the event to the one or more digital resource object categories based on information available in a public database.

At step 309, the token generation server 120 is configured to determine at least one magnitude value associated with each of the one or more digital resource object categories based on an evaluation of the event. In some embodiments, the magnitude value generated at step 309 corresponds to the digital token values derived at step 212 and/or digital token value augmented by a data completeness score, described at step 218. In various embodiments, the evaluation includes an analysis of one or more parameters associated with the event. The one or more parameters include at least one of an extent or a duration associated with the event. In various embodiments, determining the at least one magnitude value further includes, for a respective digital resource object category mapped to the event, determining, by the token generation server 120, one or more digital resource object sub-categories mapped to the respective digital resource object category, determining at least one parameter value associated with each of the one or more digital resource object sub-categories based on an evaluation of a data association between the respective digital resource object category and each of the one or more digital resource object sub-categories, and aggregating each determined parameter value. In some embodiments, the one or more digital resource object sub-categories are associated with a mortality projection model or a survival analysis model of an entity or a member associated with an entity.

In some embodiments, determining the at least one magnitude value further includes receiving, by the token generation server 120, one or more attribute data objects associated with the entity, determining whether each of the one or more attribute data objects is a positive or a negative modifier of the evaluation of the event, and modifying the at least one magnitude value based on the determination.

In some embodiments, the event includes a plurality of sub-events detected over a period of time. Additionally, determining the at least one magnitude value further includes for a respective digital resource object category mapped to the event, determining, by the token generation server 120, a data complete score associated with the event, where the data completeness score is based on a data completeness rating of the plurality of sub-events detected over the period of time, and modifying the at least one magnitude value based on the data completeness score. In some embodiments, the value of the aggregate digital resource object is modified by a fluctuating weight, where the fluctuating weight is determined based on a quantity of similarly classified data objects detected in the blockchain as compared to the one or more data objects.

In some embodiments, the token generation server 120 is configured to map the entity to a group entity based on a requirement to include the group entity in the blockchain with the event when entering event data, or a search of a public or private repository that includes information related to the group entity. In some embodiments, the token generation server 120 is configured to issue the aggregate digital resource object to the group entity or to the entity. In some embodiments, the aggregate digital resource object is used to reduce a plan premium, or the aggregate digital resource object is exchangeable on an exchange platform for one or more other digital resource objects. In various embodiments, the token generation server 120 is configured to generate a digital resource object for each digital resource object category mapped to the event.

In various embodiments, the user device 112 is configured so that a user (e.g., associated entity or its members) may view individual digital tokens generated (e.g., with respect to methods 200 and/or 300) for each digital token category or aggregate digital tokens generated through the display 116. For example, individual digital tokens generated for each digital token category or aggregate digital tokens that are generated are output to the user interface 118 of the display 116 so that a user can see how many tokens he or she has received. In some embodiments, the user application 114 is in communication with the token generation server 120 to receive data associated with a quantity of digital tokens that are generated for a user. The user application 114, in addition to supporting the various functionalities described above, enables a user to view digital tokens generated and received, pending status of digital tokens that are in queue to be generated, history of submission of event data, evaluation or analysis results of submitted event data, among others. The user application 114 is further in communication with the incentive application 180 to further support exchange of digital tokens for various incentives. In this respect, the user application 114 supports various functionalities that enable a user to trade digital tokens, exchange digital tokens for other digital tokens or currencies, use digital tokens to lower health premiums, among other functionalities.

In various embodiments, the generated digital tokens are exchangeable for various incentives. In some embodiments, the incentive application 180 is configured to host one or more applications that enable associated entities or their members to exchange their digital tokens for various incentives, in accordance with an exchange system outlined below. This exchange system is configured to enable members or associated entities to exchange digital tokens they receive for other digital tokens or digital currencies, or for other resources or currencies. Individual members may also be able to publicly trade digital tokens they receive on external exchanges for other digital currencies or digital tokens.

In some embodiments, the incentive application 180 enables clients who are considering a new contract with a health insurance provider, to optimize the ideal set of health plans that would be mutually beneficial to the client, its members, and the health insurance provider in exchange for digital tokens generated through methods 200 or 300.

In some embodiments, the incentive application 180 enables new employers that are subscribed to a health insurance provider to buy or loan, with interest, digital tokens generated through methods 200 or 300 for existing employers, to help offset premium payments, similar to carbon offset trading. Alternatively, the incentive application 180 enables existing employers subscribed to a health insurance provider to exchange digital tokens on internal or external exchanges to offset premium payments for its employees or members.

To describe an example use case of the exchange system, the token generation server 120 is configured to receive event data from a company, where the event data corresponds to data of its employees. For each type of data received, the token generation server 120 is configured to generate and issue the company digital tokens in accordance with the steps outlined method 200. The company may be looking to open a facility in a rural area due to tax benefits designed to incentivize industrialization of an economically distressed area. The company may know that this region has a poor community health rating. To further incentivize construction of new facilities, the incentive application 180 enables the manufacturer to trade received digital tokens with another company to offset its expected high health premiums of its employees. These transactions may be kept private and anonymous.

The digital tokens that are generated in accordance with methods 200 and/or 300 not only reflect the data collected but also an analysis of that data and how it relates to member health. If fiat currencies can be said to reflect the health of a country's economic activity (e.g., GDP), the digital tokens or aggregate digital tokens that are generated may reflect the physical health of a member base.

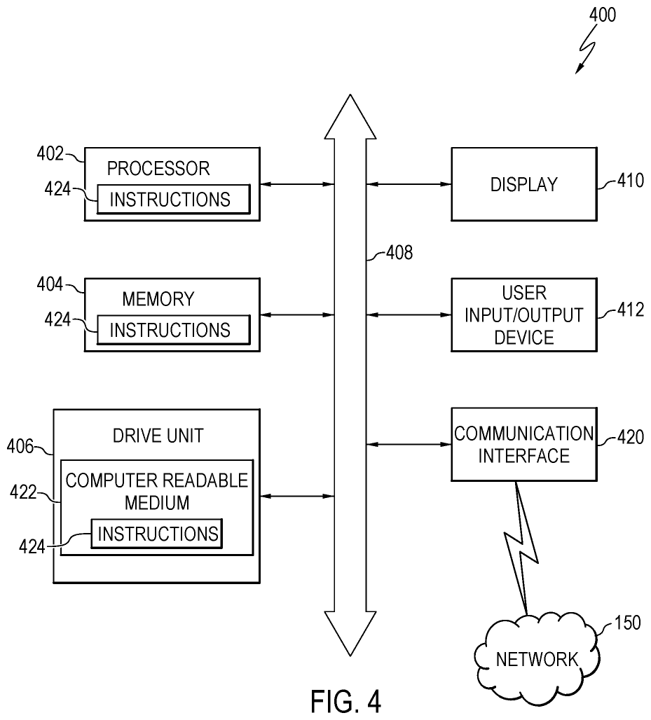

FIG. 4 depicts an example implementation of a computer system that executes techniques presented herein, according to one or more embodiments. The computer system 400 includes a set of instructions that are executed to cause the computer system 400 to perform any one or more of the methods or computer based functions disclosed herein. The computer system 400 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,” or a “server” includes one or more processors.

In a networked deployment, the computer system 400 operates in the capacity of a server or as a client user computer in a server-client user network environment, or as a peer computer system in a peer-to-peer (or distributed) network environment. The computer system 400 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 400 is implemented using electronic devices that provide voice, video, or data communication. Further, while the computer system 400 is illustrated as a single system, the term “system” shall also be taken to include any collection of systems or sub-systems that individually or jointly execute a set, or multiple sets, of instructions to perform one or more computer functions.

As illustrated in FIG. 4, the computer system 400 includes a processor 402, e.g., a central processing unit (CPU), a graphics processing unit (GPU), or both. The processor 402 is a component in a variety of systems. For example, the processor 402 is part of a standard personal computer or a workstation. The processor 402 is one or more processors, digital signal processors, application specific integrated circuits, field programmable gate arrays, servers, networks, digital circuits, analog circuits, combinations thereof, or other now known or later developed devices for analyzing and processing data. The processor 402 implements a software program, such as code generated manually (i.e., programmed).

The computer system 400 includes a memory 404 that communicates via bus 408. The memory 404 is a main memory, a static memory, or a dynamic memory. The memory 404 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 404 includes a cache or random-access memory for the processor 402. In alternative implementations, the memory 404 is separate from the processor 402, such as a cache memory of a processor, the system memory, or other memory. The memory 404 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 404 is operable to store instructions executable by the processor 402. The functions, acts, or tasks illustrated in the figures or described herein are performed by the processor 402 executing the instructions stored in the memory 404. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 400 further includes a display 410, such as a liquid crystal display (LCD), an organic light emitting diode (OLED), a flat panel display, a solid-state display, a cathode ray tube (CRT), a projector, a printer or other now known or later developed display device for outputting determined information. The display 410 acts as an interface for the user to see the functioning of the processor 402, or specifically as an interface with the software stored in the memory 404 or in the drive unit 406.

Additionally or alternatively, the computer system 400 includes an input/output device 412 configured to allow a user to interact with any of the components of the computer system 400. The input/output device 412 is a number pad, a keyboard, a cursor control device, such as a mouse, a joystick, touch screen display, remote control, or any other device operative to interact with the computer system 400.

The computer system 400 also includes the drive unit 406 implemented as a disk or optical drive. The drive unit 406 includes a computer-readable medium 422 in which one or more sets of instructions 424, e.g. software, is embedded. Further, the sets of instructions 424 embodies one or more of the methods or logic as described herein. The sets of instructions 424 resides completely or partially within the memory 404 and/or within the processor 402 during execution by the computer system 400. The memory 404 and the processor 402 also include computer-readable media as discussed above.

In some systems, computer-readable medium 422 includes the set of instructions 424 or receives and executes the set of instructions 424 responsive to a propagated signal so that a device connected to network 150 communicates voice, video, audio, images, or any other data over the network 150. Further, the sets of instructions 424 are transmitted or received over the network 150 via the communication port or interface 420, and/or using the bus 408. The communication port or interface 420 is a part of the processor 402 or is a separate component. The communication port or interface 420 is created in software or is a physical connection in hardware. The communication port or interface 420 is configured to connect with the network 150, external media, the display 410, or any other components in the computer system 400, or combinations thereof. The connection with the network 150 is a physical connection, such as a wired Ethernet connection, or is established wirelessly as discussed below. Likewise, the additional connections with other components of the computer system 400 are physical connections or are established wirelessly. The network 150 can alternatively be directly connected to the bus 408.

While the computer-readable medium 422 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 422 is non-transitory, and may be tangible.

The computer-readable medium 422 includes a solid-state memory such as a memory card or other package that houses one or more non-volatile read-only memories. The computer-readable medium 422 is a random-access memory or other volatile re-writable memory. Additionally or alternatively, the computer-readable medium 422 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

The computer system 400 is connected to the network 150. The network 150 defines one or more networks including wired or wireless networks. The network 150 includes, for example, the Internet, intranets, extranets, wide area networks (WANs), local area networks (LANs), wired networks, wireless networks, cable networks, satellite networks, or other suitable networks, etc., or any combination of two or more such networks. The network 150 is configured to couple one computing device to another computing device to enable communication of data between the devices. The network 150 is generally enabled to employ any form of machine-readable media for communicating information from one device to another. The network 150 includes communication methods by which information travels between computing devices. The network 150 can be divided into sub-networks. The sub-networks allow access to all of the other components connected thereto or the sub-networks restrict access between the components. The network 150 can be regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, and HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention are practiced without these specific details. In other instances, well-known methods, structures and techniques have not been shown in detail in order not to obscure an understanding of this description.

Thus, while there has been described what are believed to be the preferred embodiments of the invention, those skilled in the art will recognize that other and further modifications are made thereto without departing from the spirit of the invention, and it is intended to claim all such changes and modifications as falling within the scope of the invention. For example, any formulas given above are merely representative of procedures that may be used. Functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

Other embodiments of the disclosure will be apparent to those skilled in the art from consideration of the specification and practice of the invention disclosed herein. It is intended that the specification and examples be considered as examples only, with a true scope and spirit of the invention being indicated by the following claims.

The present disclosure furthermore relates to the following aspects.

Example 1. A computer-implemented method, comprising: detecting, by one or more processors, a presence of an event written to a blockchain as one or more data objects, the event being associated with an entity; determining, by the one or more processors, one or more digital resource object categories to be mapped to the event based on one or more factors; determining, by the one or more processors, at least one magnitude value associated with each of the one or more digital resource object categories based on an evaluation of the event, the evaluation including an analysis of one or more parameters associated with the event; and generating, by the one or more processors, an aggregate digital resource object based on each determined magnitude value.

Example 2. The computer-implemented method of example 1, wherein the event is written to the blockchain based on a consensus mechanism.

Example 3. The computer-implemented method of any of the preceding examples, wherein the one or more digital resource object categories include one or more health-related categories.

Example 4. The computer-implemented method of any of the preceding examples, wherein the one or more factors include: a predefined mapping of known events to the one or more digital resource object categories; and/or an existing association of the event to the one or more digital resource object categories based on information available in a public database.

Example 5. The computer-implemented method of any of the preceding examples, wherein the one or more parameters include at least one of an extent or a duration associated with the event.

Example 6. The computer-implemented method of any of the preceding examples, wherein determining the at least one magnitude value further includes: for a respective digital resource object category mapped to the event, determining, by the one or more processors, one or more digital resource object sub-categories mapped to the respective digital resource object category; determining, by the one or more processors, at least one parameter value associated with each of the one or more digital resource object sub-categories based on an evaluation of a data association between the respective digital resource object category and each of the one or more digital resource object sub-categories; and aggregating, by the one or more processors, each determined parameter value.

Example 7. The computer-implemented method of example 6, wherein the one or more digital resource object sub-categories are associated with a mortality projection model or a survival analysis model.

Example 8. The computer-implemented method of any of the preceding examples, wherein the one or more digital resource object categories are associated with a health determinant model.

Example 9. The computer-implemented method of any of the preceding examples, wherein determining the at least one magnitude value further includes: receiving, by the one or more processors, one or more attribute data objects associated with the entity; determining, by the one or more processors, whether each of the one or more attribute data objects is a positive or a negative modifier of the evaluation of the event; and modifying, by the one or more processors, the at least one magnitude value based on the determination.

Example 10. The computer-implemented method of any of the preceding examples, wherein the event includes a plurality of sub-events detected over a period of time, and wherein determining the at least one magnitude value further includes: for a respective digital resource object category mapped to the event, determining, by the one or more processors, a data completeness score associated with the event, the data completeness score being based on a data completeness rating of the plurality of sub-events detected over the period of time; and modifying, by the one or more processors, the at least one magnitude value based on the data completeness score.

Example 11. The computer-implemented method of any of the preceding examples, wherein a value of the aggregate digital resource object is modified by a fluctuating weight, the fluctuating weight being determined based on a quantity of similarly classified data objects detected in the blockchain as compared to the one or more data objects.

Example 12. The computer-implemented method of any of the preceding examples, further comprising mapping, by the one or more processors, the entity to a group entity based on: a requirement to include the group entity in the blockchain with the event; or a search of a public or private repository that includes information related to the group entity.

Example 13. The computer-implemented method of example 12, further comprising issuing, by the one or more processors, the aggregate digital resource object to the group entity or to the entity.

Example 14. The computer-implemented method of any of the preceding examples, wherein: the aggregate digital resource object is used to reduce a plan premium; or the aggregate digital resource object is exchangeable on an exchange platform for one or more other digital resource objects.

Example 15. The computer-implemented method of any of the preceding examples, further comprising: generating, by the one or more processors, a digital resource object for each digital resource object category mapped to the event.

Example 16. A system, comprising: at least one memory having processor-readable instructions stored therein; and at least one processor configured to access the at least one memory and execute the processor-readable instructions to perform operations, the operations comprising: detecting a presence of an event written to a blockchain as one or more data objects, the event being associated with an entity; determining one or more digital resource object categories to be mapped to the event based on one or more factors; determining at least one magnitude value associated with each of the one or more digital resource object categories based on an evaluation of the event, the evaluation including an analysis of one or more parameters associated with the event; and generating an aggregate digital resource object based on each determined magnitude value.

Example 17. The system of example 16, wherein the event is written to the data store based on a consensus mechanism.

Example 18. The system of any of Examples 16-17, wherein determining the at least one magnitude value further includes: for a respective digital resource object category mapped to the event, determining one or more digital resource object sub-categories mapped to the respective digital resource object category; determining at least one parameter value associated with each of the one or more digital resource object sub-categories based on an evaluation of a data association between the respective digital resource object category and each of the one or more digital resource object sub-categories; and aggregating each determined parameter value.

Example 19. The system of example 18, wherein the one or more digital resource object categories are associated with a health determinant model, and wherein the one or more digital resource object sub-categories are associated with a mortality projection model or a survival analysis model.

Example 20. A non-transitory computer-readable medium storing a set of instructions that, when executed by at least one processor, cause the at least one processor to perform operations comprising: detecting a presence of an event written to a blockchain as one or more data objects, the event being associated with an entity; determining one or more digital resource object categories to be mapped to the event based on one or more factors; determining at least one magnitude value associated with each of the one or more digital resource object categories based on an evaluation of the event, the evaluation including an analysis of one or more parameters associated with the event; and generating an aggregate digital resource object based on each determined magnitude value.

What is claimed is:

1. A computer-implemented method, comprising:
detecting, by one or more processors, a presence of an event written to a blockchain as one or more data objects, the event being associated with an entity;
determining, by the one or more processors, one or more digital resource object categories to be mapped to the event based on one or more factors;
determining, by the one or more processors, at least one magnitude value associated with each of the one or more digital resource object categories based on an evaluation of the event, the evaluation including an analysis of one or more parameters associated with the event; and
generating, by the one or more processors, an aggregate digital resource object based on each determined magnitude value.

2. The computer-implemented method of claim 1, wherein the event is written to the blockchain based on a consensus mechanism.

3. The computer-implemented method of claim 1, wherein the one or more digital resource object categories include one or more health-related categories.

4. The computer-implemented method of claim 1, wherein the one or more factors include:
a predefined mapping of known events to the one or more digital resource object categories; and/or
an existing association of the event to the one or more digital resource object categories based on information available in a public database.

5. The computer-implemented method of claim 1, wherein the one or more parameters include at least one of an extent or a duration associated with the event.

6. The computer-implemented method of claim 1, wherein determining the at least one magnitude value further includes:
for a respective digital resource object category mapped to the event,
determining, by the one or more processors, one or more digital resource object sub-categories mapped to the respective digital resource object category;
determining, by the one or more processors, at least one parameter value associated with each of the one or more digital resource object sub-categories based on an evaluation of a data association between the respective digital resource object category and each of the one or more digital resource object sub-categories; and
aggregating, by the one or more processors, each determined parameter value.

7. The computer-implemented method of claim 6, wherein the one or more digital resource object sub-categories are associated with a mortality projection model or a survival analysis model.

8. The computer-implemented method of claim 1, wherein the one or more digital resource object categories are associated with a health determinant model.

9. The computer-implemented method of claim 1, wherein determining the at least one magnitude value further includes:
receiving, by the one or more processors, one or more attribute data objects associated with the entity;
determining, by the one or more processors, whether each of the one or more attribute data objects is a positive or a negative modifier of the evaluation of the event; and
modifying, by the one or more processors, the at least one magnitude value based on the determination.

10. The computer-implemented method of claim 1, wherein the event includes a plurality of sub-events detected over a period of time, and wherein determining the at least one magnitude value further includes:
for a respective digital resource object category mapped to the event,
determining, by the one or more processors, a data completeness score associated with the event, the data completeness score being based on a data completeness rating of the plurality of sub-events detected over the period of time; and
modifying, by the one or more processors, the at least one magnitude value based on the data completeness score.

11. The computer-implemented method of claim 1, wherein a value of the aggregate digital resource object is modified by a fluctuating weight, the fluctuating weight being determined based on a quantity of similarly classified data objects detected in the blockchain as compared to the one or more data objects.

12. The computer-implemented method of claim 1, further comprising mapping, by the one or more processors, the entity to a group entity based on:
a requirement to include the group entity in the blockchain with the event; or
a search of a public or private repository that includes information related to the group entity.

13. The computer-implemented method of claim 12, further comprising issuing, by the one or more processors, the aggregate digital resource object to the group entity or to the entity.

14. The computer-implemented method of claim 1, wherein:
the aggregate digital resource object is used to reduce a plan premium; or
the aggregate digital resource object is exchangeable on an exchange platform for one or more other digital resource objects.

15. The computer-implemented method of claim 1, further comprising:
generating, by the one or more processors, a digital resource object for each digital resource object category mapped to the event.

16. A system, comprising:
at least one memory having processor-readable instructions stored therein; and
at least one processor configured to access the at least one memory and execute the processor-readable instructions to perform operations, the operations comprising:
detecting a presence of an event written to a blockchain as one or more data objects, the event being associated with an entity;
determining one or more digital resource object categories to be mapped to the event based on one or more factors;
determining at least one magnitude value associated with each of the one or more digital resource object categories based on an evaluation of the event, the evaluation including an analysis of one or more parameters associated with the event; and
generating an aggregate digital resource object based on each determined magnitude value.

17. The system of claim 16, wherein the event is written to the data store based on a consensus mechanism.

18. The system of claim 16, wherein determining the at least one magnitude value further includes:
for a respective digital resource object category mapped to the event,
determining one or more digital resource object sub-categories mapped to the respective digital resource object category;
determining at least one parameter value associated with each of the one or more digital resource object sub-categories based on an evaluation of a data association between the respective digital resource object category and each of the one or more digital resource object sub-categories; and
aggregating each determined parameter value.

19. The system of claim 18, wherein the one or more digital resource object categories are associated with a health determinant model, and wherein the one or more digital resource object sub-categories are associated with a mortality projection model or a survival analysis model.

20. A non-transitory computer-readable medium storing a set of instructions that, when executed by at least one processor, cause the at least one processor to perform operations comprising:
detecting a presence of an event written to a blockchain as one or more data objects, the event being associated with an entity;
determining one or more digital resource object categories to be mapped to the event based on one or more factors;
determining at least one magnitude value associated with each of the one or more digital resource object categories based on an evaluation of the event, the evaluation including an analysis of one or more parameters associated with the event; and
generating an aggregate digital resource object based on each determined magnitude value.
